(19) United States
(12) Patent Application Publication
(10) Pub. No.: US 2005/0113868 A1
(43) Pub. Date: May 26, 2005

(54) Device, with electrospun fabric, for a percutaneous transluminal procedure, and methods thereof

(76) Inventors: Carol A. Devellian, Topsfield, MA (US); David R. Widomski, Wakefield, MA (US)

Correspondence Address: TESTA, HURWITZ & THIBEAULT, LLP, HIGH STREET TOWER, 125 HIGH STREET, BOSTON, MA, 02110, US

(21) Appl. No.: 10/874,968

(22) Filed: Jun. 22, 2004

Related U.S. Application Data
(60) Provisional application No. 60/523,628, filed on Nov. 20, 2003.

(51) Int. Cl. A61B 17/08

(52) U.S. Cl. 606/213; 606/157; 606/151; 606/215

(57) ABSTRACT

The invention relates to an occluder for a percutaneous transluminal procedure. In one embodiment, the occluder includes an overall support structure and a plurality of occlusion shells connected to the overall support structure. At least one occlusion shell includes an electrospun fabric.

### CROSS-REFERENCE TO RELATED APPLICATION

This application incorporates by reference, and claims priority to and the benefit of, U.S. provisional application Ser. No. 60/523,628, which was filed on Nov. 20, 2003.

### TECHNICAL FIELD

The invention generally relates to devices and related methods for closing cardiac openings. More particularly, the invention features an occluder, which includes an electrospun fabric, for the percutaneous transluminal closure of a patent foramen ovale or a left atrial appendage.

### BACKGROUND

The human heart is divided into four compartments or chambers. The left and right atria are located in the upper portion of the heart and the left and right ventricles are located in the lower portion of the heart. The left and right atria are separated from each other by a muscular wall, the intraatrial septum, while the ventricles are separated by the intraventricular septum.

Either congenitally or by acquisition, abnormal openings, holes, or shunts can occur between the chambers of the heart or between the great vessels, causing blood to inappropriately flow therethrough. Such deformities are usually congenital and originate during fetal life when the heart forms from a folded tube into a four chambered, two unit system. The septal deformities result from the incomplete formation of the septum, or muscular wall, between the chambers of the heart and can cause significant problems.

One such deformity or defect, a patent foramen ovale, is a persistent, one-way, usually flap-like opening in the wall between the right atrium and left atrium of the heart. Since left atrial pressure is normally higher than right atrial pressure, the flap typically stays closed. Under certain conditions, however, right atrial pressure exceeds left atrial pressure, creating the possibility for right to left shunting that can allow blood clots to enter the systemic circulation. This is particularly problematic for patients who are prone to forming venous thrombus, such as those with deep vein thrombosis or clotting abnormalities.

Moreover, certain patients are prone to atrial arrhythmias (i.e., abnormal heart rhythms which can cause the heart to pump less effectively). In a common such abnormality, atrial fibrillation, the two upper chambers of the heart (i.e., the left atria and the right atria), quiver instead of beating effectively. Because the atria do not beat and empty cleanly during atrial fibrillation, blood can stagnate on the walls and form clots that can then pass through the heart and into the brain, causing a stroke or a transient ischemic attack. These clots typically form in a cul-de-sac in the heart called the left artrial appendage due to its tendency to have low or stagnant flow.

Nonsurgical (i.e., percutaneous) closure of a patent foramen ovale, as well as similar cardiac openings such as an atrial septal defect or a ventricular septal defect, and obliteration of a left atrial appendage are possible using a variety of mechanical devices. These devices typically consist of a metallic structural framework with a scaffold material attached thereto. Currently available closure devices, however, are often complex to manufacture, are inconsistent in performance, require a technically complex implantation procedure, lack anatomic conformability, and lead to complications (e.g., thrombus formation, chronic inflammation, residual leaks, perforations, fractures, and cardiac conduction system disturbances).

Improved devices and related methods for closing cardiac openings, such as, for example, a patent foramen ovale, and for obliterating cardiac cul-de-sacs, such as, for example, a left atrial appendage, are, therefore, needed.

### SUMMARY OF THE INVENTION

The present invention features a device and related methods for percutaneously closing a cardiac opening, such as, for example, a patent foramen ovale, an atrial septal defect, or a ventricular septal defect, and for percutaneously obliterating a cardiac cul-de-sac, such as, for example, a left atrial appendage. A scaffold material of the inventive device includes, at least in part, an electrospun fabric. In a preferred embodiment, the electrospun fabric is an electrospun matrix of polymer fibers. In some embodiments, the polymer fibers are combined with, or are themselves exclusively, a substance for stimulating tissue growth and, therefore, closure of a cardiac opening. In some other embodiments, the polymer fibers are combined with, or are themselves exclusively, an anti-thrombotic material. As a result of this structure, the aforementioned disadvantages associated with the devices known in the art are minimized or eliminated.

In general, in one aspect, the invention features an occluder for a percutaneous transluminal procedure. The occluder includes an overall support structure and a plurality of occlusion shells connected to the overall support structure. At least one of the occlusion shells includes an electrospun fabric.

Various embodiments of this aspect of the invention include the following features. The electrospun fabric can be an electrospun matrix of polymer fibers. The polymer fibers can include a substance for stimulating tissue growth (e.g., collagen or a growth factor) and/or an anti-thrombotic material (e.g., heparin). In other embodiments, the overall support structure includes a metal, or, alternatively, a bioresorbable polymer, such as, for example, a polylactic acid.

In yet another embodiment, the overall support structure includes both a proximal support structure and a distal support structure. In one embodiment, the proximal support structure and the distal support structure together form a clip. In another embodiment, the proximal support structure includes a plurality of outwardly extending proximal arms and the distal support structure includes a plurality of outwardly extending distal arms. The proximal support structure can connect to a proximal occlusion shell and the distal support structure can connect to a distal occlusion shell.

In another aspect, the invention features an occluder for a percutaneous transluminal procedure. The occluder includes an overall support structure and at least one occlusion shell connected to the overall support structure. The at least one occlusion shell includes an electrospun fabric. In a particular embodiment, the at least one occlusion shell includes a substance for stimulating tissue growth.

In yet another aspect, the invention features a method for percutaneous transluminal closure of a cardiac opening in a patient. The method includes inserting an occluder into a heart of the patient and positioning the occluder at least partially within the cardiac opening to substantially occlude the cardiac opening. The occluder includes an overall support structure and at least one occlusion shell connected to the overall support structure. The at least one occlusion shell includes an electrospun fabric.

In some embodiments of this aspect of the invention, the cardiac opening is, for example, a patent foramen ovale, an atrial septal defect, or a ventricular septal defect. In another embodiment, the overall support structure of the occluder includes a proximal support structure and a distal support structure. The proximal support structure connects to a proximal occlusion shell and the distal support structure connects to a distal occlusion shell. A portion of the overall support structure is positioned within the cardiac opening, while the proximal occlusion shell and the distal occlusion shell are positioned on different sides of the cardiac opening.

In still another aspect, the invention features a method for percutaneous transluminal obliteration of a cardiac cul-de-sac in a patient. The method includes inserting an occluder into a heart of the patient and positioning the occluder at least partially within the cardiac cul-de-sac to substantially obliterate the cardiac cul-de-sac. The occluder includes an overall support structure and at least one occlusion shell connected to the overall support structure. The at least one occlusion shell includes an electrospun fabric. In one embodiment of this aspect of the invention, the cardiac cul-de-sac is a left atrial appendage.

In a further aspect, the invention features a method for making an occluder for a percutaneous transluminal procedure. The method includes providing an overall support structure and connecting a plurality of occlusion shells to the overall support structure. At least one of the plurality of occlusion shells includes an electrospun fabric.

In various embodiments of this aspect of the invention, the at least one occlusion shell that includes the electrospun fabric is, for example, sewn, laminated, or glued to the overall support structure and coated with the electrospun fabric by electrospinning a matrix of polymer fibers onto the at least one occlusion shell as a coating. Alternatively, in another embodiment, to connect the at least one occlusion shell that includes the electrospun fabric to the overall support structure, a matrix of polymer fibers is electrospun directly onto the overall support structure.

In some embodiments, producing the elecrospun fabric by electrospinning a matrix of polymer fibers includes discharging a jet of polymer fibers. A direction of travel of the discharged jet of polymer fibers may be controlled by applying, for example, an electric field, a magnetic field, or an electromagnetic field across at least a portion of a length of the discharged jet.

The foregoing and other objects, aspects, features, and advantages of the invention will become more apparent from the following description and from the claims.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 is a cutaway view of a heart illustrating a patent foramen ovale.

FIG. 2 is a partial cross-sectional view of another heart illustrating a left atrial appendage.

FIG. 3 is a schematic top view of an occluder according to an illustrative embodiment of the invention.

FIG. 4 is a schematic cross-sectional view of the illustrative occluder shown in FIG. 3.

FIG. 5 is a schematic top view of an occluder according to another illustrative embodiment of the invention.

FIG. 6 is a schematic side view of the illustrative occluder shown in FIG. 5.

FIG. 7 is a schematic perspective view of an occluder according to another illustrative embodiment of the invention.

FIG. 8 is a schematic perspective view of an occluder for obliterating a cardiac cul-de-sac according to an illustrative embodiment of the invention.

FIG. 9 is a schematic perspective view of an occluder for obliterating a cardiac cul-de-sac according to another illustrative embodiment of the invention.

FIG. 10 is a schematic view of an apparatus for electrospinning a matrix of polymer fibers according to an illustrative embodiment of the invention.

FIGS. 11A-11E illustrate the stages, according to an illustrative embodiment of the invention, for delivering an occluder to an anatomical site in the body of a patient.

### DESCRIPTION

The present invention features an occluder for closing cardiac openings, such as, for example, a patent foramen ovale, and for obliterating cardiac cul-de-sacs, such as, for example, a left atrial appendage. The occluder includes a structural framework and at least one occlusion shell. In one embodiment, a fabric is electrospun directly onto the structural framework of the occluder to form the at least one occlusion shell in its entirety. In another embodiment, a pre-existing occlusion shell is first connected (e.g., sewn, laminated, or glued) to the structural framework of the occluder and then enhanced by electrospinning a fabric thereon.

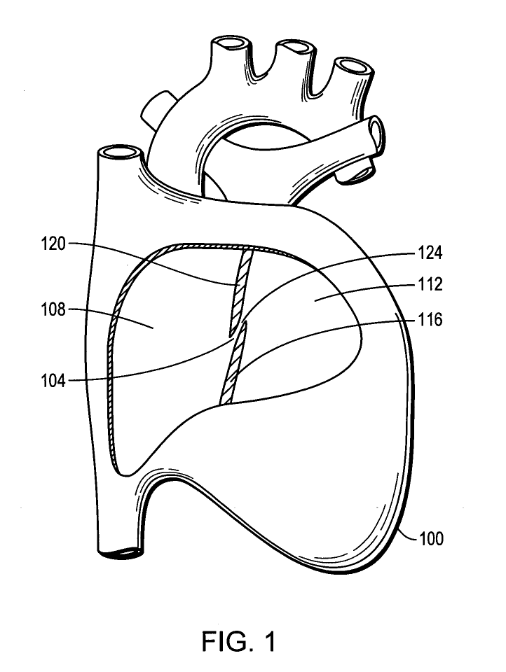

FIG. 1 depicts a cutaway view of a heart 100. The heart 100 includes a septum 104 that divides a right atrium 108 from a left atrium 112. The septum 104 includes a septum primum 116 and a septum secundum 120. An exemplary cardiac opening, a patent foramen ovale 124, that is to be corrected by the occluder of the present invention is located between the septum primum 116 and the septum secundum 120. The patent foramen ovale 124 provides an undesirable fluid communication between the right atrium 108 and the left atrium 112 and, under certain conditions, allows for the shunting of blood from the right atrium 108 to the left atrium 112. If the patent foramen ovale 124 is not closed or obstructed in some manner, a patient can be placed at a higher risk for an embolic stroke.

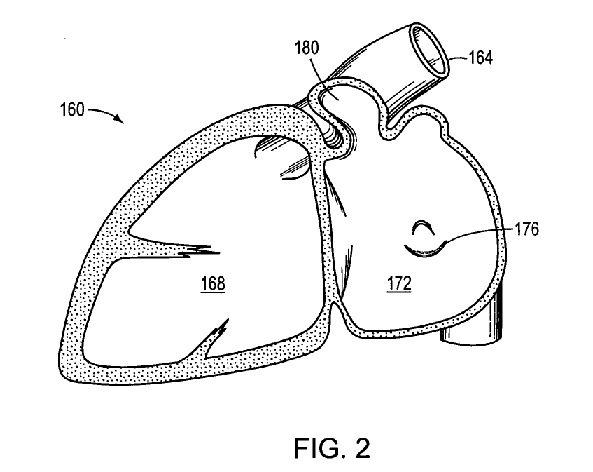

FIG. 2 depicts a partial cross-sectional view of another heart 160. The heart 160 includes an aorta 164, a left ventricle 168, a left atrium 172, and a fossa ovalis 176. The heart 160 also includes an exemplary cardiac cul-de-sac, a left atrial appendage 180, that is to be obliterated by the occluder of the present invention. Under certain conditions, clots may form in the left atrial appendage 180. If the left atrial appendage 180 is not closed or obstructed in some manner, a patient is placed at high risk of having the clots pass through the heart 160 and into the brain, causing a stroke or a transient ischemic attack.

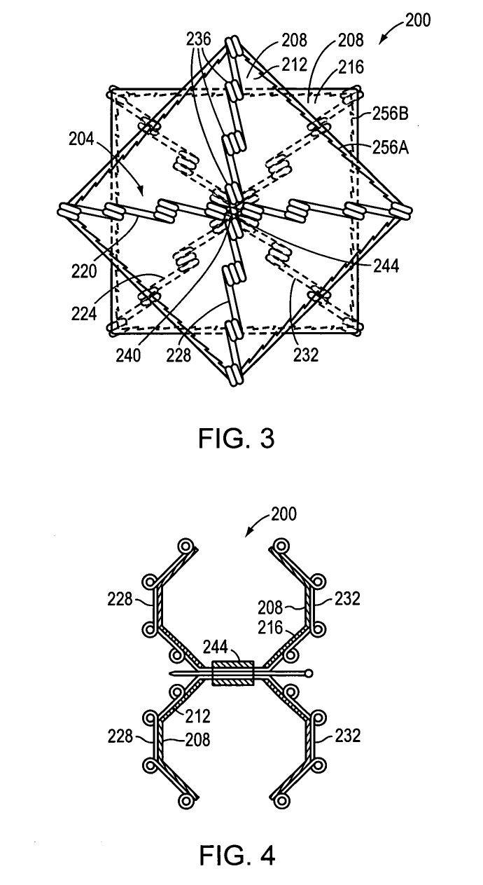

FIG. 3 depicts an occluder 200, capable of being used for the percutaneous transluminal closure of a cardiac opening, according to an illustrative embodiment of the invention. The occluder 200 includes an overall support structure 204 and at least one occlusion shell 208 that is connected to the overall support structure 204. For example, the occluder 200 includes two occlusion shells 208 that are connected to the overall support structure 204: a proximal occlusion shell 212 (i.e., an occlusion shell that is closest to a physician when the physician is implanting the occluder 200 into a body of a patient) and an opposite, distal occlusion shell 216. As described below, at least one occlusion shell 208 is coated with an electrospun fabric, or, alternatively, is itself made entirely of the electrospun fabric.

In one embodiment, the overall support structure 204 includes a proximal support structure 220, for connecting to and supporting the proximal occlusion shell 212, and a distal support structure 224, for connecting to and supporting the distal occlusion shell 216. Both the proximal support structure 220 and the distal support structure 224 can include any number of outwardly extending arms, typically four or more outwardly extending arms, to support each of their respective occlusion shells 212, 216. In one embodiment, as shown in FIG. 3, the proximal support structure 220 includes four outwardly extending proximal arms 228 and the distal support structure 224 similarly includes four outwardly extending distal arms 232.

In one embodiment, each outwardly extending arm is resiliently biased as a result of including three or more resilient coils 236 radially spaced from a center point 240. Alternatively, other resilient support structures could be used. In one embodiment, the proximal support structure 220 and the distal support structure 224 are mechanically secured together by wire 244. Alternatively, other means, such as, for example, laser welding, may be used to secure the proximal support structure 220 to the distal support structure 224.

FIG. 4 depicts a cross-sectional view of the occluder 200 illustrated in FIG. 3. Four arms 228, 232, are shown.

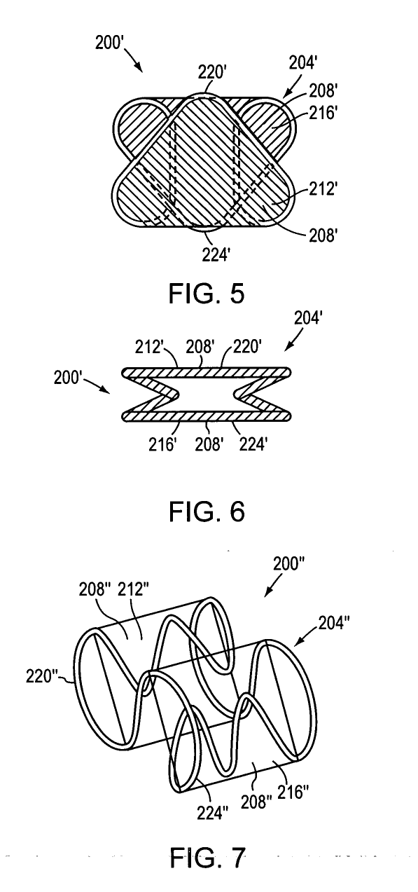

FIGS. 5 and 6 depict an occluder 200′ according to another illustrative embodiment of the invention. An overall support structure 204′, which includes a proximal support structure 220′, for supporting a proximal occlusion shell 212′, and a distal support structure 224′, for supporting a distal occlusion shell 216′, is shaped as a clip.

FIG. 7 depicts an occluder 200″ according to yet another illustrative embodiment of the invention. Again, an overall support structure 204″ forms a clip and includes a proximal support structure 220″, for supporting a proximal occlusion shell 212″, and a distal support structure 224″, for supporting a distal occlusion shell 216″.

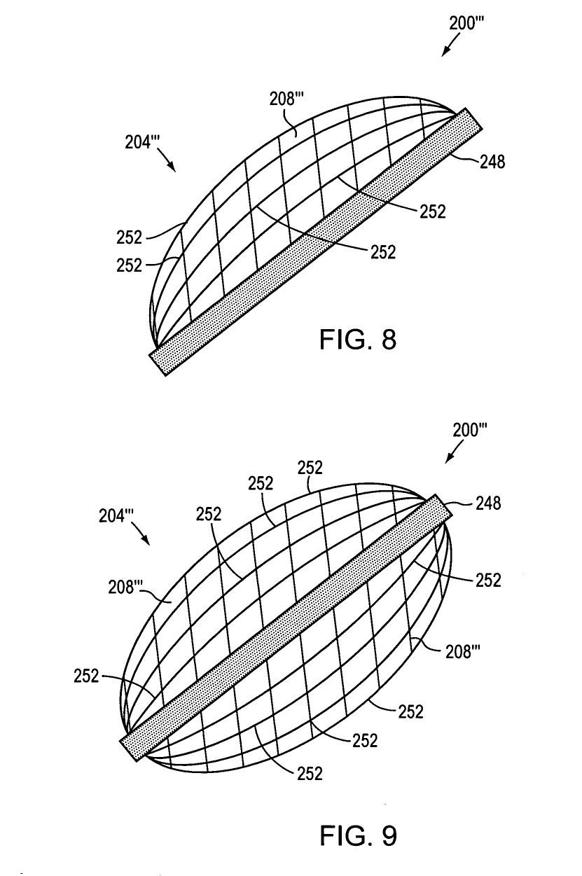

FIGS. 8 and 9 depict an occluder 200′″ according to still another illustrative embodiment of the invention. As shown, an overall support structure 204′″ includes a central attachment mechanism 248 and a plurality of legs 252 for connecting to and supporting an occlusion shell 208′″. The legs 252 can be connected to the central attachment mechanism 248 so as to define a substantially hemispherical outer surface, as shown in FIG. 8, or, alternatively, so as to define a substantially spherical outer surface, as shown in FIG. 9. The occlusion shell 208′″ can be connected to the legs 252 so as to cover the entire substantially hemispherical outer surface, illustrated in FIG. 8, so as to cover the entire substantially spherical outer surface, illustrated in FIG. 9, or so as to cover any portions thereof.

The occluders 200, 200′, and 200″ depicted in FIGS. 3-7 are, in various embodiments, particularly useful in closing cardiac openings such as a patent foramen ovale, an atrial septal defect, or a ventricular septal defect. The occluder 200′″ depicted in FIGS. 8-9 is, in various embodiments, particularly useful for obliterating cardiac cul-de-sacs such as a left atrial appendage.

As would be readily apparent to one skilled in the art, the overall support structure 204 can assume any shape or configuration and is not limited to the exemplary embodiments discussed above.

In one embodiment, the overall support structure 204 is fabricated from metal, such as, for example, stainless steel, a nickel-titanium alloy (e.g., Nitinol, which is manufactured by Nitinol Devices and Components of Freemont, Calif.), or a nickel-cobalt-chromium-molybdenum alloy (e.g., MP35N®, which is manufactured by SPS Technologies, Inc. of Jenkintown, Pa.). The metal may be capable of corroding in the body of a patient. Alternatively, the metal may be corrosion resistant. In other embodiments, the overall support structure 204 is fabricated from bioresorbable or biodegradeable polymers, such as, for example, polylactic acid, polyglycolic acid, polydioxanone, polyethylene glycol, and polycapralactone. Moreover, the overall support structure 204 can be flexible and resilient. It can, therefore, as explained below, be collapsed within a sheath for delivery to an anatomical site in the body of a patient and thereafter, upon deployment, be expanded to occlude a cardiac opening.

In accordance with the present invention, at least one occlusion shell 208 is made, either entirely or in part, from an electrospun fabric, such as, for example, an electrospun matrix of polymer fibers.

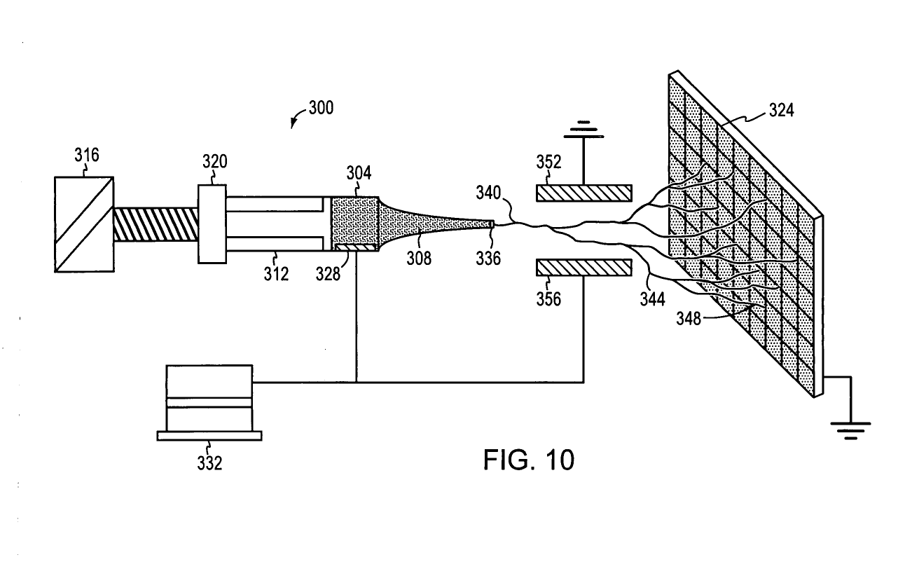

FIG. 10 depicts an exemplary apparatus 300 either for making, in its entirety, an occlusion shell 208 for an occluder 200, or for enhancing the occlusion shell 208, according to an illustrative embodiment of the invention. As shown, the apparatus 300 includes, in one embodiment, a tube (e.g., a glass tube or a polymer tube) 304, such as, for example, a pipette. A fluid 308, such as, for example, a polymer solution or a polymer melt, is contained within the tube 304. In one embodiment, the apparatus 300 also includes a syringe 312, which is connected to the tube 304 and which is used to advance the fluid 308 through the tube 304. Moreover, the apparatus 300 can include a metering pump 316, which can be attached, for example, to a plunger 320 of the syringe 312 and used to generate a constant pressure on the syringe 312, thereby ensuring a constant flow of the fluid 308 through the tube 304. Alternatively, in another embodiment, the tube 304 is simply tilted a few degrees below the horizontal, depending on the viscosity of the fluid 308, thus creating a constant flow rate of the fluid 308 through the tube 304.

Also depicted in FIG. 10 is a collector 324 for the electrospun fabric, which is produced as described below. In one embodiment, the occlusion shell 208 is made in its entirety from electrospun fabric. In such a case, the collector 324 is the overall support structure 204 of the occluder 200 and a matrix of polymer fibers is electrospun directly onto the overall support structure 204 to form the occlusion shell 208. Alternatively, in another embodiment, a pre-existing occlusion shell 208 is coated with an electrospun fabric. In one such embodiment, the pre-existing occlusion shell 208 is first attached to the overall support structure 204 of the occluder 200 and then enhanced by electrospinning a matrix of polymer fibers onto the pre-existing occlusion shell 208 (i.e., the collector 324 is the pre-existing occlusion shell 208, which has been attached to the overall support structure 204 of the occluder 200). In this latter case, and with reference to FIG. 3 for example, the pre-existing occlusion shell 208 can be sewn, as at 256A, 256B, with any commonly used suture material (e.g., a polyester suture), to the overall support structure 204. Alternatively, the pre-existing occlusion shell 208 can be laminated, glued, or attached by, for example, hooks or thermal welding to the overall support structure 204. In one embodiment, for example, the pre-existing occlusion shell 208 can be laminated to the overall support structure 204, such that the overall support structure 204 is encapsulated entirely within the pre-existing occlusion shell 208. The pre-existing occlusion shell 208 may be made from, for example, a polyester fabric (e.g., a woven or knitted polyester fabric), a polyvinyl sponge (e.g., Ivalon®, manufactured by Unipoint Industries, Inc. of High Point, N.C.), an expanded polytetrafluoroethylene (ePTFE) material, or a metal mesh.

Referring again to FIG. 10, in one embodiment, an electrode 328, attached to a high voltage source 332, is immersed into the fluid 308 of the tube 304 and used to provide the fluid 308 with an electric charge. The collector 324 is, for its part, grounded, as illustrated. For example, in one embodiment, the metallic overall support structure 204 of the occluder 200 is grounded. As such, an electric field is generated between the fluid 308 and the collector 324. By providing the fluid 308 with an electric charge, mutual charge repulsion causes a force directly opposite to the surface tension of the fluid 308. As the intensity of the electric field is increased, a hemispherical surface of the fluid 308 at a tip 336 of the tube 304 elongates to form a conical shape, known to those skilled in the art as a Taylor cone. By continuing to increase the electric field, a critical value is finally attained. At this critical value, the repulsive electrostatic force overcomes the surface tension of the fluid 308 and a charged jet 340 of fluid 308 is ejected from the tip of the Taylor cone in the direction of the grounded collector 324. As the jet 340 travels towards to the grounded collector 324, it undergoes a whipping process, producing elongated polymer fibers 344 of very small diameter. Where the fluid 308 is, for example, a polymer solution, the solvent evaporates during the whipping process, leaving behind a charged matrix 348 of polymer fibers 344 on the grounded collector 324. Where the fluid 308 is, for example, a polymer melt, the discharged jet 340 solidifies into a charged polymer fiber 344 as it travels in the air towards the collector 324, and is randomly collected on the collector 324 to form the matrix 348 of polymer fibers 344. In accordance with the invention, polymer fibers 344 in the range of nanometers to a few microns can be produced.

In one embodiment, during the electrospinning procedure described above, the collector 324 is rotated or moved in the X, Y, and/or Z directions of a Cartesian coordinate system, such that the charged polymer fibers 344 are disposed about the surface of the collector 324. In another embodiment, the apparatus 300 is rotated or moved in the X, Y, and/or Z directions of a Cartesian coordinate system, such that the charged polymer fibers 344 are disposed about the surface of the collector 324. In yet another embodiment, a first electrode place 352 can be, as illustrated, positioned above at least a portion of the discharged jet 340 and a second electrode plate 356 can be positioned below at least a portion of the discharged jet 340. The electrode plates 352, 356 can apply another electric field across at least a portion of the length of the discharged jet 340. The direction of travel of the discharged jet 340 can thereby be controlled and, as such, so can the resulting pattern of the matrix 348 of polymer fibers 344 on the collector 324. To provide the electric field, the second electrode plate 356 can be, for example, attached to the high voltage source 332 and the first electrode plate 352 can be grounded, as shown. Alternatively, an electromagnetic field or a magnetic field can be applied across at least a portion of the length of the discharged jet 340 so as to control the direction of travel of the discharged jet 340 and, as such, the resulting pattern of the matrix 348 of polymer fibers 344 on the collector 324.

In one embodiment, the occlusion shell 208, which is either entirely formed by or, alternatively, enhanced by the electrospinning process described above, is non-porous and prevents the passage of fluids that are intended to be retained by the implantation of the occluder 200. Alternatively, in another embodiment, the occlusion shell 208 is porous to facilitate tissue ingrowth into the occlusion shell 208, thereby promoting occlusion of the cardiac opening.

In one embodiment, the polymer, before being used in the electrospinning process described above, is combined with a substance for stimulating tissue growth (e.g., a physiological reactive chemical). Alternatively, in another embodiment, the polymer is itself a substance for stimulating tissue growth. The growth stimulating substance can be, for example, a collagen. In another embodiment, the growth stimulating substance is a growth factor, such as a vascular endothelial growth factor, a basic fibro growth factor, or an angiogenic growth factor. In yet another embodiment, the growth stimulating substance is a pharmacological agent for stimulating tissue growth, such as, for example, cells or genes. Alternatively, in still another embodiment, the growth stimulating substance is an irritant for encouraging an inflammatory response, such as, for example, cod liver oil, cotton seed oil, or alcohol.

In yet another embodiment, the polymer is combined, before being used in the electrospinning process, with a chemical compound and/or material for enhancing radiopacity. Exemplary chemical compounds that may be used to increase radiopacity include, but are not limited to, barium sulfate, calcium sulfate, bismuth oxide, and iodine.

In still another embodiment, heparin is ionically or covalently bonded to the occlusion shell 208, and/or to the electrospun fabric forming the whole or a part of the occlusion shell 208, to render it non-thrombogenic. Alternatively, proteins or cells are applied to the occlusion shell 208 and/or the electrospun fabric to render it non-thrombogenic and/or to accelerate the healing process.

A variety of polymers can be electrospun (so long as they can be dissolved in an appropriate solvent or solvent mixture to make a concentrated solution and the molecular weight is high enough, or, alternatively, so long as the polymer melt can be used) to produce, or enhance, as described above, the occlusion shell 208 of the occluder 200. Examples of such polymers include, but are not limited to, polyimides, polyamic acid, polyetherimide, Nylon 6 & Nylon 66, polyaramid, poly-gamma-benzyl-glutamate, poly (p-phenylene terephthalamide), polybenzimidazole (PBI), Ultem 1000 (polyetherimide), nylon 6-polyimide, polyacrylonitrile, polyethylene terephtalate (PET), polypropylene, nylon, polyaniline, polyhydroxybutyrate-valerate, polyethylene oxide (PEO), polynaphthalene terephthalate (PEN), polybutylene terephthalate (PBT), styrene-butadiene rubber (SBR), Shell's Kraton (SBS), polystyrene (PS), mesophase pitch, polyvinyl chloride (PVC), polyvinyl alcohol (PVA), expanded polytetrafluoroethylene (ePTFE), naturally occurring biopolymers, and bioresorbable polyesters, including, but not limited to, polylactide, polyglycolide, tyrosine derived polycarbonate, and blends and copolymers thereof.

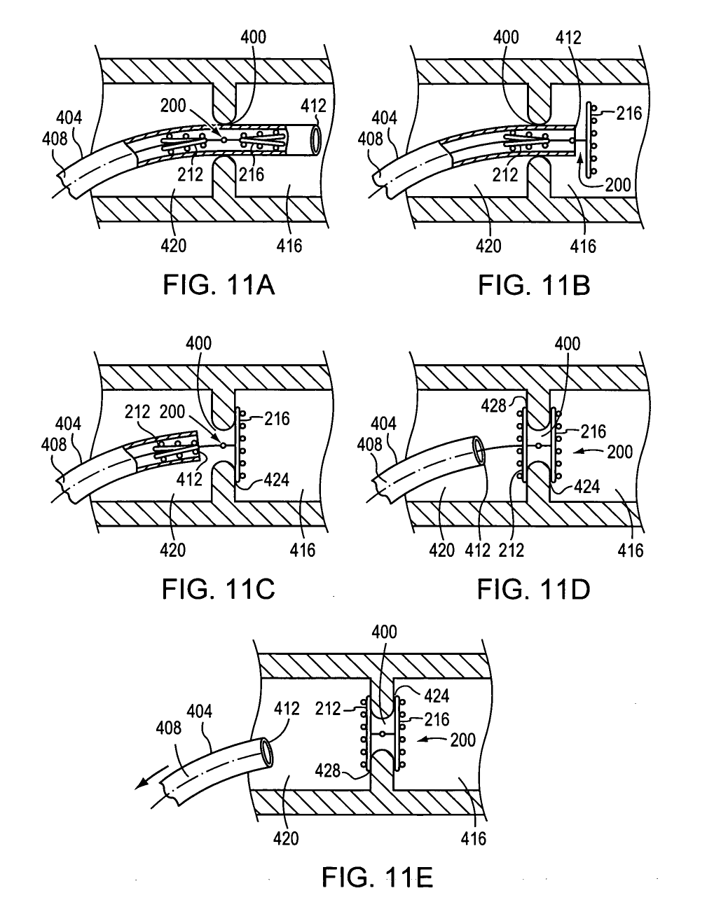

FIGS. 11A-11E depict the stages for delivering the occluder 200, according to an illustrative embodiment of the invention, percutaneously to an anatomical site in the body of a patient for closing a cardiac opening 400, such as, for example, a patent foramen ovale, an atrial septal defect, or a ventricular septal defect. Referring to FIG. 11A, a sheath 404 is first inserted into the cardiac opening 400, as is typically performed by one skilled in the art. The occluder 200 is then loaded into a lumen 408 of the sheath 404 and advanced throughout the lumen 408 until positioned at a distal end 412 of the sheath 404. Referring to FIG. 11B, the distal occlusion shell 216 of the occluder 200 is then released into a distal heart chamber 416 through the distal end 412 of the sheath 404. The distal occlusion shell 216 opens automatically and resiliently. The sheath 404 is then pulled back into a proximal heart chamber 420, as illustrated in FIG. 11C, to seat the distal occlusion shell 216 against a distal wall surface 424 of the cardiac opening 400. The cardiac opening 400 is thereby occluded from the distal side. As shown in FIG. 11D, the sheath 404 is then further withdrawn a sufficient distance to allow the proximal occlusion shell 212 to be released from the distal end 412 of the sheath 404. The proximal occlusion shell 212 opens automatically and resiliently to lie against a proximal surface 428 of the cardiac opening 400, occluding the cardiac opening 400 from the proximal side. The sheath 404 is then withdrawn from the patient's body, leaving behind the opened occluder 200. As shown in FIG. 11E, the occlusion shells 212, 216 are positioned on either side of the cardiac opening 400 and the occluder 200 is permanently implanted within the body of the patient.

In another embodiment, where, for example, the left atrial appendage requires obliteration as therapy for stroke, the stages for delivering an occluder (e.g., the occluder 200′″ described above with reference to FIGS. 8 and 9) to the left atrial appendage differ from the stages immediately described above. Specifically, a physician only performs the stage illustrated with reference to FIG. 11A. That is, the physician first inserts a sheath 404 into the lumen of the left atrial appendage, as is typically performed by one skilled in the art, and then loads the occluder 200′″, in a collapsed position, into the lumen 408 of the sheath 404. The occluder 200′″ is then advanced throughout the lumen 408 until positioned at the distal end 412 of the sheath 404. Because the anatomical structure of the left atrial appendage differs from that of a patent foramen ovale, an atrial septal defect, or a ventricular septal defect, the operator then simply places the occluder 200′″ into the left atrial appendage. Placed as such, the occluder 200′″ expands automatically and resiliently to permanently close off the left atrial appendage.

Variations, modifications, and other implementations of what is described herein will occur to those of ordinary skill in the art without departing from the spirit and the scope of the invention. The invention is not to be defined only by the preceding illustrative description.

1. An occluder for a percutaneous transluminal procedure, comprising:
an overall support structure; and
a plurality of occlusion shells connected to the overall support structure, wherein at least one of the occlusion shells comprises an electrospun fabric.

2. The occluder of claim 1, wherein the electrospun fabric comprises an electrospun matrix of polymer fibers.

3. The occluder of claim 2, wherein the polymer fibers comprise a substance for stimulating tissue growth.

4. The occluder of claim 3, wherein the substance for stimulating tissue growth comprises collagen.

5. The occluder of claim 3, wherein the substance for stimulating tissue growth comprises a growth factor.

6. The occluder of claim 2, wherein the polymer fibers comprise an anti-thrombotic material.

7. The occluder of claim 6, wherein the anti-thrombotic material comprises heparin.

8. The occluder of claim 1, wherein the overall support structure comprises a metal.

9. The occluder of claim 1, wherein the overall support structure comprises a bioresorbable polymer.

10. The occluder of claim 9, wherein the bioresorbable polymer comprises polylactic acid.

11. The occluder of claim 1, wherein the overall support structure comprises a proximal support structure and a distal support structure.

12. The occluder of claim 11, wherein the proximal support structure and the distal support structure form a clip.

13. The occluder of claim 11, wherein the proximal support structure comprises a plurality of outwardly extending proximal arms and the distal support structure comprises a plurality of outwardly extending distal arms.

14. The occluder of claim 11, wherein the proximal support structure connects to a proximal occlusion shell and the distal support structure connects to a distal occlusion shell.

15. An occluder for a percutaneous transluminal procedure, comprising:
an overall support structure; and
at least one occlusion shell, connected to the overall support structure, comprising an electrospun fabric and a substance for stimulating tissue growth.

16. A method for percutaneous transluminal closure of a cardiac opening in a patient, comprising:
inserting an occluder into a heart of the patient, the occluder comprising:
an overall support structure; and
at least one occlusion shell connected to the overall support structure and comprising an electrospun fabric; and
positioning the occluder at least partially within the cardiac opening to substantially occlude the cardiac opening.

17. The method of claim 16, wherein the overall support structure of the occluder comprises a proximal support structure and a distal support structure, the proximal support structure connecting to a proximal occlusion shell and the distal support structure connecting to a distal occlusion shell, and wherein positioning the occluder at least partially within the cardiac opening comprises positioning a portion of the overall support structure within the cardiac opening and positioning the proximal occlusion shell and the distal occlusion shell on different sides of the cardiac opening.

18. The method of claim 16, wherein the cardiac opening is a patent foramen ovale.

19. The method of claim 16, wherein the cardiac opening is an atrial septal defect.

20. The method of claim 16, wherein the cardiac opening is a ventricular septal defect.

21. A method for percutaneous transluminal obliteration of a cardiac cul-de-sac in a patient, comprising:
inserting an occluder into a heart of the patient, the occluder comprising:
an overall support structure; and
at least one occlusion shell connected to the overall support structure and comprising an electrospun fabric; and
positioning the occluder at least partially within the cardiac cul-de-sac to substantially obliterate the cardiac cul-de-sac.

22. The method of claim 21, wherein the cardiac cul-de-sac is a left atrial appendage.

23. A method for making an occluder for a percutaneous transluminal procedure, comprising:
providing an overall support structure; and
connecting a plurality of occlusion shells to the overall support structure, wherein at least one of the plurality of occlusion shells comprises an electrospun fabric.

24. The method of claim 23, wherein the at least one occlusion shell comprising the electrospun fabric is connected to the overall support structure by electrospinning a matrix of polymer fibers directly onto the overall support structure.

25. The method of claim 23, wherein the at least one occlusion shell comprising the electrospun fabric is connected to the overall support structure by sewing at least one occlusion shell to the overall support structure, and by electrospinning a matrix of polymer fibers onto the at least one occlusion shell as a coating.

26. The method of claim 23, wherein the at least one occlusion shell comprising the electrospun fabric is connected to the overall support structure by laminating at least one occlusion shell to the overall support structure, and by electrospinning a matrix of polymer fibers onto the at least one occlusion shell as a coating.

27. The method of claim 23, wherein the at least one occlusion shell comprising the electrospun fabric is connected to the overall support structure by gluing at least one occlusion shell to the overall support structure, and by electrospinning a matrix of polymer fibers onto the at least one occlusion shell as a coating.

28. The method of claim 23 further comprising producing the electrospun fabric by electrospinning a matrix of a polymer fibers.

29. The method of claim 28, wherein electrospinning the matrix of polymer fibers comprises discharging a jet of polymer fibers.

30. The method of claim 29, wherein a direction of travel of the discharged jet of polymer fibers is controlled by applying an electric field across at least a portion of a length of the discharged jet.

31. The method of claim 29, wherein a direction of travel of the discharged jet of polymer fibers is controlled by applying a magnetic field across at least a portion of a length of the discharged jet.

32. The method of claim 29, wherein a direction of travel of the discharged jet of polymer fibers is controlled by applying an electromagnetic field across at least a portion of a length of the discharged jet.
